(19) United States
(12) Patent Application Publication
(10) Pub. No.: US 2018/0353152 A1
(43) Pub. Date: Dec. 13, 2018

(54) SOUND DETECTION APPARATUS, SYSTEM AND METHOD

(71) Applicant: Ann and Robert H. Lurie Children's Hospital of Chicago, Chicago, IL (US)

(72) Inventor: Jagjit Singh Teji, Oak Brook, IL (US)

(21) Appl. No.: 16/003,587

(22) Filed: Jun. 8, 2018

Related U.S. Application Data
(60) Provisional application No. 62/517,350, filed on Jun. 9, 2017.

(51) Int. Cl. A61B 7/02; G08B 21/18; H04R 1/40; H04R 1/08; H04R 3/04; A61B 5/00

(52) U.S. Cl. CPC A61B 7/026; G08B 21/182; H04R 1/406; H04R 1/083; H04R 3/04; A61B 2503/06; A61B 5/742; A61B 5/7405; H04R 2201/403; A61B 2503/04; A61B 5/746

(57) ABSTRACT

A sound detection system for detecting heart or lung sounds is disclosed, comprising a sound detection apparatus, a signal processor, and an indicator. A representative sound detection apparatus includes an optional flexible material layer; and an array of a plurality of sound sensors to generate a plurality of sound signals, and a signal output interface. A signal processor includes a memory storing reference data; and a processor adapted to receive the plurality of sound signals, to compare the received plurality of sound signals with the reference data, and when a variance or difference between one or more of the received plurality of sound signals and the reference data is greater than a predetermined threshold, to generate a corresponding command signal to the indicator to provide a warning signal indication.

### CROSS-REFERENCE TO A RELATED APPLICATION

This application is a nonprovisional of and claims the benefit of and priority to U.S. Provisional Patent Application No. 62/517,350, filed Jun. 9, 2017, inventor Jagjit Singh Teji, titled “Sound Detection Apparatus, System and Method”, which is commonly assigned herewith, and all of which is hereby incorporated herein by reference in its entirety with the same full force and effect as if set forth in its entirety herein.

### FIELD OF THE INVENTION

The present invention, in general, relates to medical devices, and more particularly, to a sound detection apparatus, system and method for detecting heart or lung sounds of a human subject such as an infant, a child or an adult.

### BACKGROUND OF THE INVENTION

Patients may have non-invasive monitoring for their lungs or heart, and may also need auxiliary equipment to assist processes such as breathing. Auscultation, however, is extremely difficult when a patient is connected to a conventional or high frequency mechanical ventilator, making it even more difficult to decipher sounds from the heart and lungs, and to differentiate artifacts. The present application also discloses a more efficient way to detect sounds from the lungs and the heart by arranging a plurality of sound sensors for sound detection from different areas at the same time.

X-rays and blood gases are also routinely required to establish maintenance care, such as X-rays used for proper delineation of Endotracheal Tube (ETT) placement, slipped ETT, lung expansion and heart size, and also for diagnoses such as pneumothorax and pulmonary edema, while blood gas measurements typically provide for determinations of oxygen and CO₂ levels and the pH of blood. Analysis of data in these conditions may also be difficult.

For example, non-invasive monitoring of sick or premature newborns in the neonatal intensive care unit (“NICU”) is a problem since newborns are too small to install many monitoring devices for precise and accurate determinations. A need remains, therefore, for an apparatus, system and method to provide continuous or periodic real time monitoring of lungs and heart sounds. In addition to such real time monitoring, such an apparatus, system and method should also provide for remote real time monitoring of lungs and heart sounds.

### SUMMARY OF THE INVENTION

As discussed in greater detail below, the representative apparatus, system and method provide for noninvasive, remote, accurate, real time monitoring of heart and lung sounds. The monitoring may be continuous or periodic. The representative apparatus and system are comparatively unobtrusive, portable, convenient and easy to use for a treating physician, a nurse, a technician, other medical personnel, while nonetheless being comparatively or sufficiently accurate to obtain meaningful results and actionable information, with a comparatively fast detection time.

A representative sound detection apparatus is disclosed for detecting heart or lung sounds of a human subject, comprising: a flexible material layer; and a plurality of sound sensors coupled to the flexible material layer.

In a representative embodiment, a sound detection apparatus may further comprise a signal output interface coupled to the plurality of sound sensors. For example, the signal output interface may comprise: a signal output coupling; and a plurality of wires coupled to the plurality of sound sensors and to the signal output coupling.

In a representative embodiment, a sound detection apparatus may further comprise an adhesive layer coupled to the flexible material layer.

In a representative embodiment, the plurality of sound sensors are arranged in an array on or within the flexible material layer. For example, the array of the plurality of sound sensors may have an X-shaped configuration of the plurality of sound sensors or symmetrical inverted Y-shaped configurations of the plurality of sound sensors.

In a representative embodiment, the flexible material layer has a flat form factor and is dimensioned to correspond to a size of the human subject. In another representative embodiment, the flexible material layer is X-shaped. In another representative embodiment, the flexible material layer is wearable.

A representative sound detection apparatus is disclosed for detecting heart or lung sounds of a human subject, comprising: a plurality of sound sensors arranged in an array; and a signal output interface coupled to the plurality of sound sensors.

In a representative embodiment, a sound detection apparatus may further comprise a housing comprising a flexible material layer, wherein the plurality of sound sensors are arranged in the array on or within the flexible material layer, and wherein the plurality of wires are arranged on or within the flexible material layer.

A sound detection system for detecting heart or lung sounds of a human subject is also disclosed, comprising a sound detection apparatus, an indicator, and a signal processor. For such a representative embodiment, for example, the sound detection apparatus may comprise: a flexible material layer; an array of a plurality of sound sensors coupled to the flexible material layer, the plurality of sound sensors to generate a plurality of sound signals, and a signal output interface coupled to the plurality of sound sensors.

In a representative embodiment, an indicator may be a visual display, a visual monitor to display an image of a detected area, and/or an auditory speaker to generate a warning sound.

In a representative embodiment, a signal processor is coupled to the signal output interface and to the indicator, with the signal processor comprising: a memory storing reference data; and a processor coupled to the indicator, to the memory and to the plurality of sound sensors, the processor adapted to receive the plurality of sound signals, to compare the received plurality of sound signals with the reference data, and when a variance or difference between one or more of the received plurality of sound signals and the reference data is greater than a predetermined threshold, to generate a corresponding command signal to the indicator to provide a warning signal indication.

In a representative embodiment, the signal processor may further comprise: a filter coupled to the plurality of sound sensors to generate a plurality of filtered sound signals; an amplifier coupled to the filter to receive the plurality of filtered sound signals and generate a plurality of amplified sound signals; and an analog-to-digital converter coupled to the processor and coupled to the amplifier, the analog-to-digital converter to receive the plurality of amplified sound signals and generate a plurality of digital sound signals and provide the plurality of digital sound signals to the processor to form the received plurality of sound signals.

In another representative embodiment, the memory further stores the plurality of digital sound signals.

In another representative embodiment, the sound detection system may further comprise: a user interface coupled to the processor, the user interface to receive user input; wherein the indicator is adapted to display one or more different types of output in response to user input.

A method for detecting heart or lung sounds of a human subject, is also disclosed, comprising: using a sensor array, detecting a plurality of sounds from a predetermined region of the human subject; generating a plurality of sound signals in response to the detected plurality of sounds; using a processor, comparing the plurality of sound signals to reference data; and generating a warning signal indication when a variance or difference between the plurality of sound signals and the reference data is greater than a predetermined threshold.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1 is a plan view of a first sound detection system and a first sound detection apparatus.

FIG. 2 is a flow chart of a sound detection method.

FIG. 3 is a plan view of the first sound detection system coupled to an infant on a front, anterior side.

FIG. 4 is a rear view of the first sound detection system coupled to an infant on a rear, posterior side.

FIG. 5 is a view of a second sound detection system coupled to a child on a front side.

FIG. 6 is a plan view of a second sound detection apparatus having a first flexible material layer.

FIG. 7 is a back view of the second sound detection apparatus coupled to an infant on a rear side.

FIG. 8 is an isometric view of the second sound detection apparatus.

FIG. 9 is a detailed block diagram of the first sound detection system and the second sound detection system.

FIG. 10 is a plan view of a second sound detection apparatus having a second flexible material layer.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways. Also, it is to be understood that the phraseology and terminology employed herein, as well as the abstract included below, are for the purposes of description and should not be regarded as limiting.

Reference will now be made in detail to the present representative embodiments of the present application, examples of which are illustrated in the accompanying drawings. Wherever possible, the same reference numbers are used in the drawings and the description to refer to the same or like parts.

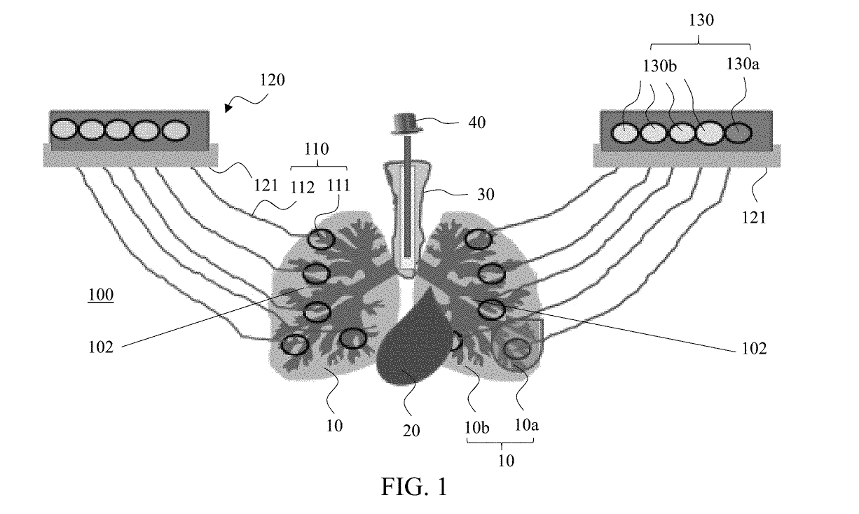

FIG. 1 is a plan view of a first sound detection system 100 and a first sound detection apparatus 110. Referring to FIG. 1, a sound detection system 100 comprises a first sound detection apparatus 110, a signal processor 120, an indicator 130, and optionally, a user interface 140. For purpose of explanation, FIG. 1 shows the positions of the first sound detection apparatus 110 with the sound sensors 111 arranged over what would be corresponding positions of the lungs 10, heart 20, trachea 30, with a ventilator 40 in place, it being understood (and as shown in greater detail below) that the sensors 111 are actually placed on the skin of the subject at such corresponding locations. As shown in FIG. 1, the first sound detection apparatus 110 comprises a plurality of sound sensors 111 and signal output interface 240 (partially illustrated only, showing a plurality of wires 112, without showing a signal output coupling 213 of the signal output interface 240). Sound sensors 111 are arranged in an array 102 and generate a plurality of sound signals in response to detecting a chest wall vibration or another auditory or vibrational signal, such as a breath sound or a heartbeat. In representative embodiments, the sound sensors 111 may be embodied or implemented in any of a plurality of ways, including implemented as piezoelectric sensors, laser sensors, microphones, sonic or ultrasonic receivers, or other signal or sound transducers, for example and without limitation, and all such variations are considered equivalent and within the scope of the disclosure. Also, the sound sensors 111 may be referred to equivalently as vibrational sensors, or sonic or ultrasonic sensors or receivers, also for example and without limitation.

As illustrated, a representative arrangement of sound sensors 111 comprises two inverted “Y” configurations, for purposes of example and not of limitation. A wide variety of arrangements of sound sensors 111 into an array 102 are available and within the scope of the present disclosure, such as arrays 102 which are X-shaped, straight, horizontal or irregular arrangements. The choice of the shape of the array 102 generally depends on the requirements of a user or subject, i.e., to correspond to the selected regions of the subject to be sensed, and the present disclosure is not limited thereto. By arranging sound sensors 111 as an array 102, the present application allows users to detect sounds from different areas of a subject's body at the same time. The wires 112 are coupled to the sound sensors 111 correspondingly to transfer the sound signals to the signal processor 120 (via a signal output coupling 213, not separately illustrated, such as an input/output connector). It should be noted that five sound sensors 111 and five wires 112 on each sides of the body are illustrated in FIG. 1 as an example, but the present application is not limited thereto, and the number of sound sensors 111 (and corresponding wires 112) depends on the requirements of a user or subject.

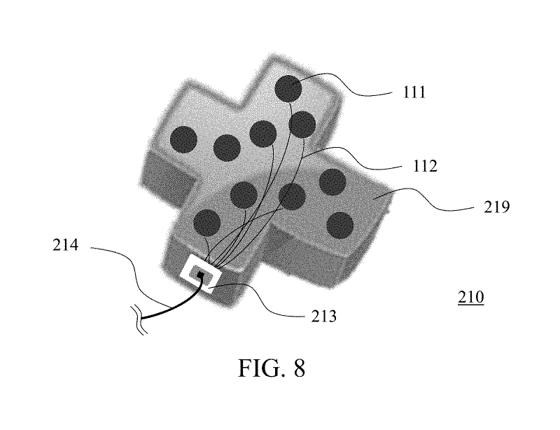

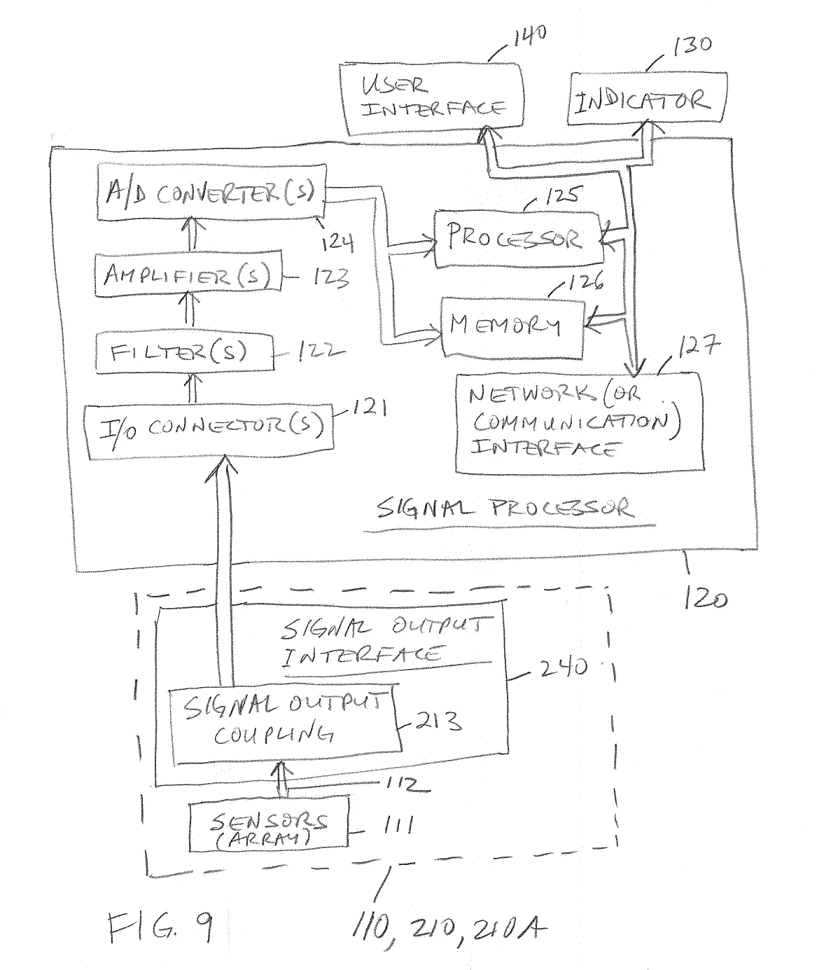

As discussed in greater detail below, in addition to other components, the signal processor 120 comprises one or more input/output (I/O) connectors 121 to receive the sound signals generated by the sound sensors 111, and the signal processor 120 will process the received data, and generate corresponding command signals, such as a command signal to the indicator 130 to display a warning indication (shown in FIG. 1 with light emitter 130a emitting a light having a first color (as a warning indication), with the other light emitters 130b emitting light of a second color (as a healthy indication, for example). The plurality of sound sensors 111 are coupled to the I/O connectors 121 of the signal processor 120 via the plurality of wires 112. Details of the signal processor 120 will be described with reference to FIGS. 8-9. The indicator 130 in the sound detection system 100 is illustrated in FIG. 1 as an array of light emitters (e.g., LEDs), such as light emitter 130a and light emitters 130b. Each of the light emitters corresponds to or represents each of the corresponding sensors 111 and corresponding regions of the subject's body. The present application, however, is not limited thereto, such as each of the light emitters can also represent more or fewer sensors 111.

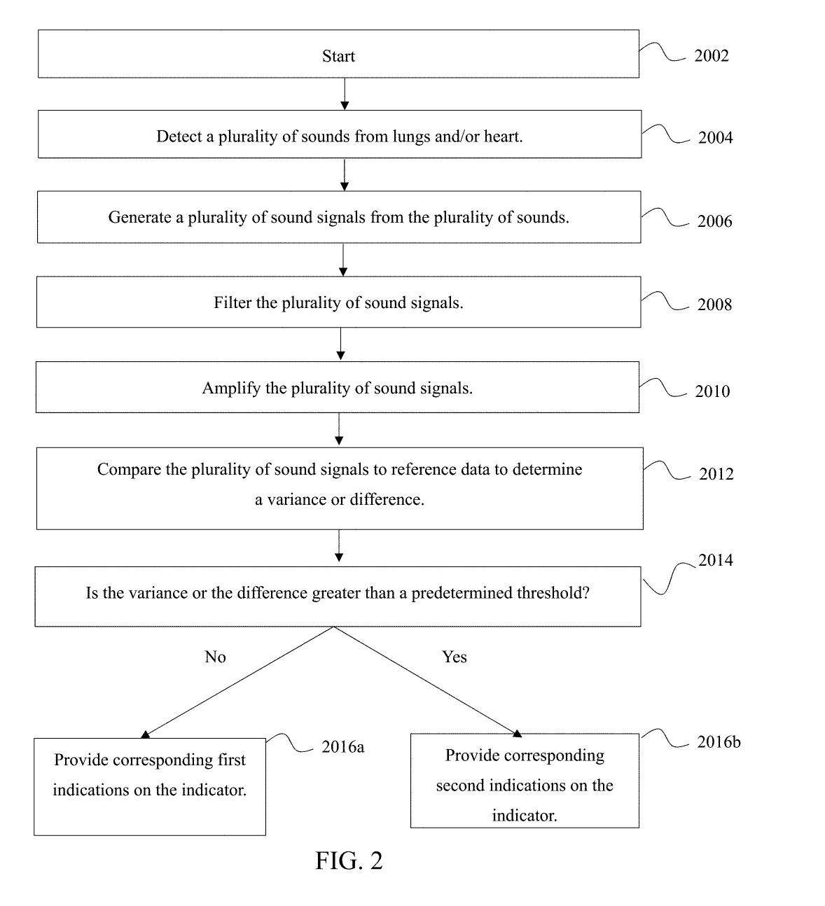

FIG. 2 is a flow chart of a sound detection method. Referring to FIGS. 1-2, preparation steps are included in a start step 2002. Specifically, a plurality of sound sensors 111 are connected to a patient. A ventilator 40 can also be installed in a trachea 30 of the patient if needed, and the present application is not so limited. The sound detection system 100 is then turned on. If the ventilator 40 is installed in the trachea 30, then the ventilator 40 is turned on as well.

After starting up the sound detection system 100, the first sound detection apparatus 110 detects a plurality of sounds from the lungs 10 and the heart 20 as shown in step 2004. The first sound detection apparatus 110 then generates a plurality of sound signals from the plurality of sounds as shown in step 2006. The sound signals are then filtered and amplified by a filter 122 and an amplifier 123 (referring to FIG. 9) as shown in step 2008 and step 2010. Details of the filter 122 and the amplifier 123 (referring to FIG. 9) will be described with reference to FIG. 9. After receiving sound signals from the first sound detection apparatus 110, the signal processor 120 compares the plurality of sound signals to reference data to determine a variance or a difference as shown in step 2012. If the variance or a difference is less than or equal to a predetermined threshold, then corresponding first indications (e.g., healthy results) are shown on an indicator 130 as shown in step 2014 and step 2016a. On the contrary, if the variance or a difference is greater than the predetermined threshold, then corresponding second indications (e.g., unhealthy results) are shown on an indicator 130 as shown in step 2014 and step 2016b, such as the warning indications mentioned above.

Referring to FIG. 1, for example, a left lobe of lung 10 has an unhealthy area 10a and a healthy area 10b. The sensor detecting sounds from the unhealthy area 10a results in the illumination of light emitter 130a. The sound sensors detecting sounds from the healthy area 10b results in the light emitters 130b not being illuminated or being illuminated in a different color, for example and without limitation. As shown in FIG. 1, the light emitter 130a lights up or emits a first color (e.g., red) as a warning signal indication while light emitters 130b remain dark or emit a second, different color (e.g., green). While the example of FIG. 1 shows that the light emitter 130a lights up when the unhealthy area 10a is detected, the present application is not limited thereto, the warning signal indication also can be other types such as flashing lights, sounds, pager signals, etc.

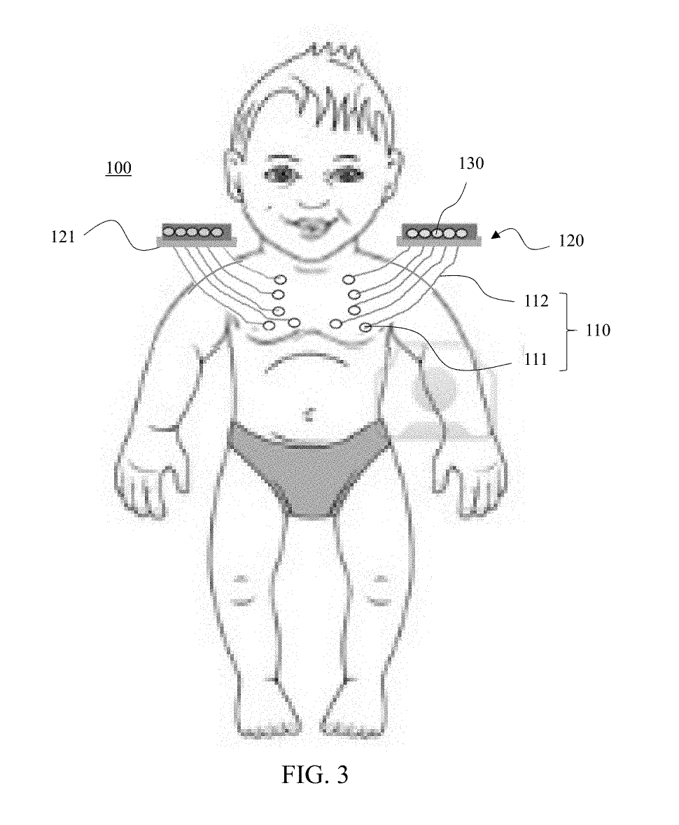

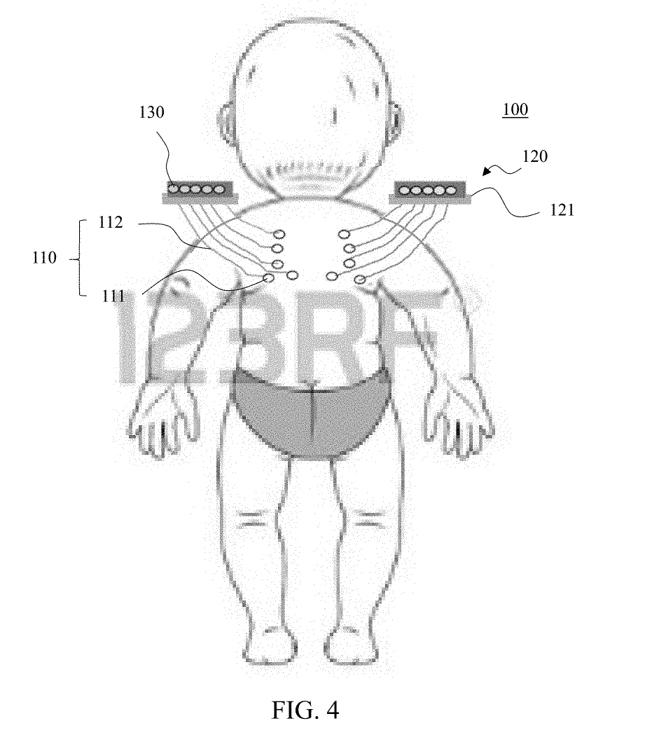

FIG. 3 is a plan view of the first sound detection system 100 coupled to an infant on a front (anterior) side. FIG. 4 is a rear view of the first sound detection system 100 coupled to an infant on a rear (posterior) side.

Referring to FIGS. 3-4, the example here shows the sound detection system 100 coupled to an infant, but the present application is not so limited, the sound detection system 100 also can be used in a child or an adult.

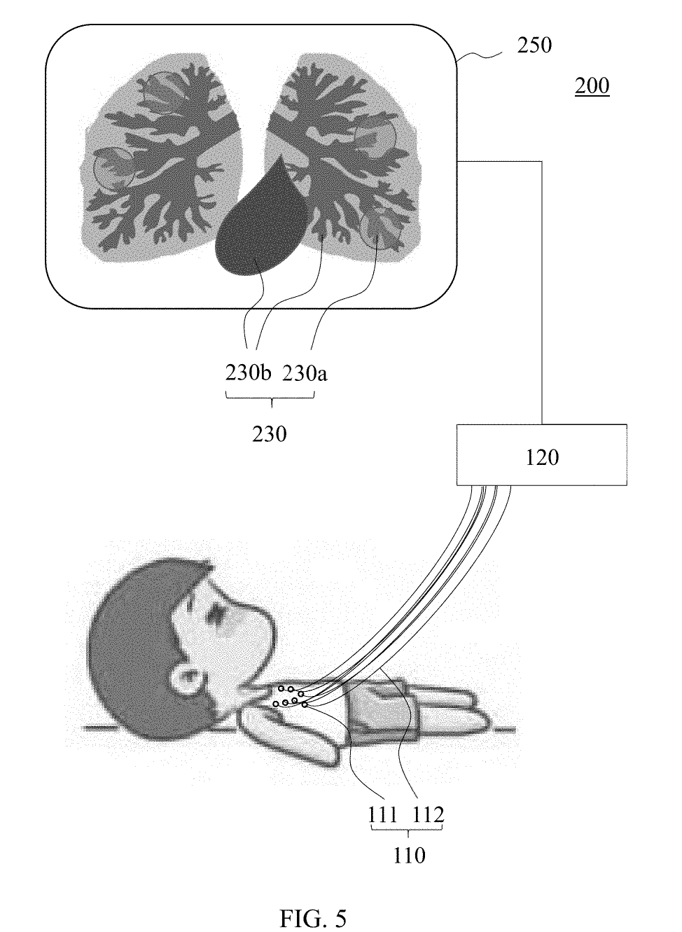

FIG. 5 is a view of a second sound detection system 200 coupled to a child on a front (anterior) side. FIG. 5 shows a second sound detection system 200 of the present application. A sound detection system 200 comprises the first sound detection apparatus 110, a signal processor 120 and an indicator 230 embodied as a monitor 250. The first sound detection apparatus 110 comprises a plurality of sound sensors 111 and a plurality of wires 112. The difference between sound detection system 100 (referring to FIG. 1) and sound detection system 200 is that a monitor 250 is used to provide the indication, showing an image of a detected area. As shown in FIG. 5, results of detection are shown on the monitor 250. The image comprises unhealthy areas 230a and healthy areas 230b, wherein unhealthy areas 230a are circled. The method for showing the unhealthy areas 230a in the present application is not so limited, the unhealthy areas 230a also can be shown by other methods, such as changing their colors.

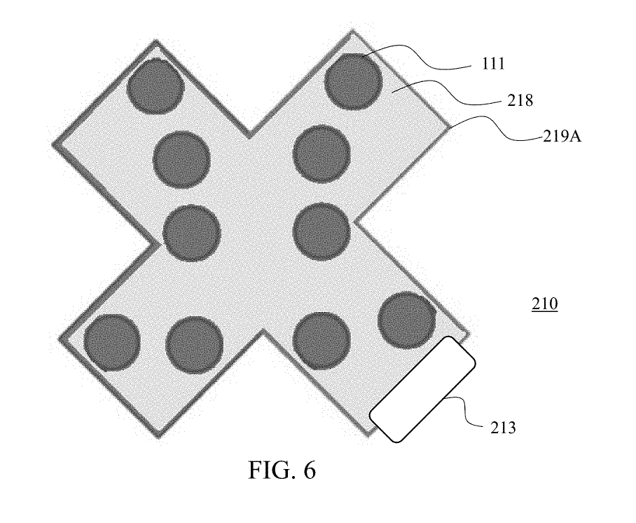

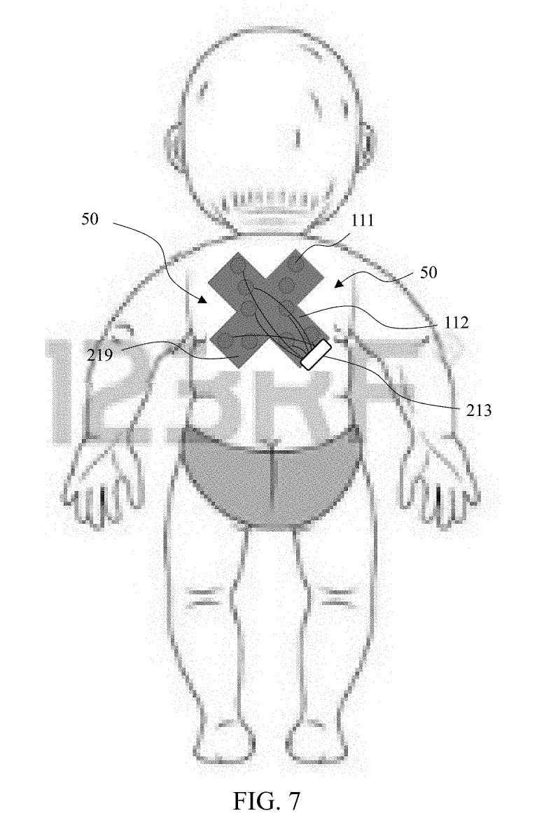

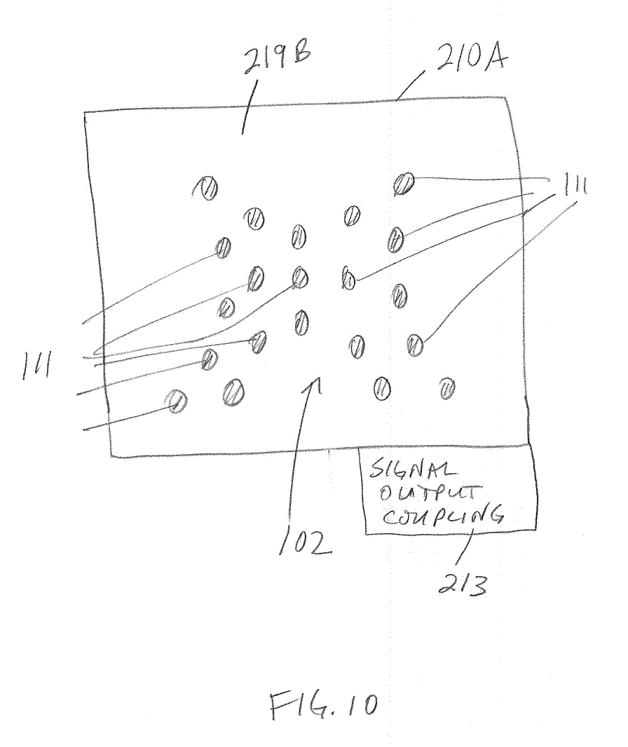

FIG. 6 is a plan view of a second sound detection apparatus 210 having a first flexible material layer 219A. FIG. 7 is a back view of the second sound detection apparatus 210 coupled to an infant on a rear side. FIG. 8 is an isometric view of the second sound detection apparatus 210A. FIG. 10 is a plan view of a second sound detection apparatus having a second flexible material layer 219B. The first flexible material layer 219A and the second flexible material layer 219B differ only in shape or configuration, and are otherwise identical. FIG. 10 also illustrates another configuration of an array 102 of sound sensors 111, such as a symmetrical “wing” or butterfly shape, as an example of the innumerable variations of array configurations which are available.

Referring to FIGS. 6 and 10, a sound detection apparatus 210 comprises a plurality of sound sensors 111, a signal output coupling 213, and a first or second flexible material layer 219A, 219B. Typically, the sound sensors 111 are coupled to the signal output coupling 213 using a plurality of wires 112. While some but not all wires 112 are illustrated in FIGS. 7 and 8 for purposes of explanation, for these embodiments, the plurality of wires 112 are typically not exposed, do not extend beyond the perimeter of the first or second flexible material layer 219A, 219B, and instead are embedded or laminated within the first or second flexible material layer 219A, 219B, so are not separately illustrated in the plan and isometric views of FIGS. 6 and 10. The plurality of sound sensors 111 may be coupled to the first or second flexible material layer 219A, 219B in any of a plurality of ways, such as using an adhesive (not separately illustrated), and any and all such variations are within the scope of the disclosure. An optional adhesive layer 218 may be coupled to the first or second flexible material layer 219A, 219B, such as to adhere the sound detection apparatus 210 to a subject. The first or second flexible material layer 219A, 219B has a flat form factor and may have any shape and size, typically dimensioned to the size of the subject, such as an adult, a child, or an infant. It should be noted that the adhesive layer 218 here is an adhesive material that may be coated on whole surface or parts of the surface of the first or second flexible material layer 219A, 219B.

In a representative embodiment, for example, the sound detection apparatus 210 may be positioned beneath a patient or subject, such as when the patient or subject is arranged on his or her back and lying in a bed, crib, or incubator. Typically, the sound detection apparatus 210 is positioned to correspond to the desired areas of examination, such as to be under the patient or subject's heart and lungs, for example. This is especially useful in a NICU, such that premature infants, for example, can be placed on their back on top of a sound detection apparatus 210, having a first or second flexible material layer 219A, 219B, which is typically soft and pliable, and is therefore similar to placing the infant on a soft blanket or a pad and is similarly comfortable to the child.

As a result, the first or second flexible material layer 219A, 219B has multiple functions, including functioning as a housing to contain the various electronic components, such as the sound sensors 111, the signal output coupling 213, and the various wires 112. The first or second flexible material layer 219A, 219B also functions as soft blanket, a pad, or other cushion for the patient.

In comparison to FIG. 6, FIG. 7 shows the second sound detection apparatus 210 without the adhesive layer 218. The first flexible material layer 219A may be wearable and X-shaped. Specifically, bones such as scapulas 50 of the patient may have influence on detection by the sound sensors 111. Therefore, with the X-shaped of first flexible material layer 219A that intentionally avoids touching those areas, the positions of sound sensors 111 can be fitted in specific locations so that the results may be more accurate. It should be noted that the material layer 219 here is intentionally made as X-shaped in order to be in compliance with the arrangement of the sound sensors 111. However, the present application is not limited thereto, the material layer 219 can be any kinds of shapes, dimensions or configurations, with the sound sensors 111 arranged in any selected array, such as an X-shaped or symmetrical inverted Y array configurations previously described. As illustrated in FIG. 7, the first flexible material layer 219A is dimensioned for an infant.

Referring to FIG. 8, further illustrates a sound detection apparatus 210 having a cable or wires 214 coupled to the signal output coupling 213 for connection to a signal processor 120. The signal output coupling 213 of the second sound detection apparatus 210 is further coupled to the I/O connector 121 (referring to FIG. 9) via cable or wires 214, as an example, and the present application is not limited thereto, the sound detection apparatus 210 also can be coupled to the I/O connector 121 via other connectors.

FIG. 9 is a block diagram of the first sound detection system 100. The sound detection system 100 comprises a signal processor 120 coupled to any of the first sound detection apparatus 110 or the second sound detection apparatus 210, and typically further comprises an indicator 130 and optionally a user interface 140.

Referring to FIG. 9, the signal processor 120 comprise a processor 125, a memory 126 coupled to the processor 125, a filter 122, an amplifier 123, an analog-to-digital (A/D) converter 124, and an I/O connector 121 coupleable to the sound sensors 111 to receive the sound signals generated by the sound sensors 111 (such as via cables or wires 214 coupled to the signal output coupling 213, which is coupled to the sound sensors 111 via wires 112).

Referring to FIG. 9, the filter 122 is coupled to the I/O connector 121 to receive the sound signals generated by the sound sensors 111 and generate a plurality of filtered sound signals. The amplifier 123 is coupled the filter 122 to receive the filtered sound signals and then generate a plurality of amplified sound signals. The A/D converter 124 is coupled to the amplifier 123 to convert the plurality of amplified sound signals to a plurality of digital sound signals. It should be noted that the filter 122 and the amplifier 123 are utilized in the exemplary embodiment, but the present application is not limited thereto. Both or one of the filter 122 and the amplifier 123 are optional and can be omitted. It should also be noted that multiple filters 122, amplifiers 123, and A/D converters 124 may be utilized, such as for separate processing of sound signals received from each sensor 111 (e.g., as separate channels).

Referring to FIG. 9, the signal processor 120 is coupled to a first or second sound detection apparatus 110, 210. The signal processor 120 further comprises a processor 125, such as a microprocessor, and a memory 126, such as a memory integrated circuit. The memory 126 is adapted to store reference data indicative of comparatively healthy heart and lung sounds, for example, and also typically stores the real time data (e.g., heart and lung sounds) of the subject or patient, i.e., the memory 126 stores the plurality of digital sound signals in addition to the reference data. The processor 125 is coupled to the memory 126, to the A/D converter 124, to the indicator 130 and an optional user interface 140. After receiving the plurality of digital sound signals from the A/D converter 124, the processor 125 is adapted to compare the received plurality of (digital) sound signals with the reference data stored in the memory 126. Although the received sound signals here is demonstrated as digital sound signals, the present application is not limited thereto, the received sound signals also can be filtered sound signals or amplified sound signals depending upon the type of processor 120 utilized (e.g., digital or analog).

Referring to FIG. 9, based upon the comparison, when a variance or difference between the received plurality of digital sound signals and the reference data is greater than a predetermined threshold, the processor 125 is adapted to generate a corresponding first command signal to the indicator 130 to provide a first indication signal, such as a warning signal indication, e.g., the lighting up of light emitters as described with reference to FIG. 1 or circling the unhealthy area on the monitor as mentioned with reference to FIG. 5, emitting a warning sound, etc. When a variance or difference between the received plurality of digital sound signals and the reference data is less than or equal to the predetermined threshold, the processor 125 is adapted to generate a corresponding second command signal to the indicator 130 to provide a second indication signal, such as the lighting up of light emitters in a second color, etc.

In addition, the sound detection system 100 may further comprise a user interface 140 coupled to the processor 125, such as for user selection of various options, such as how the indicator 130 may display the various indication signals, and so on. For example, the indicator 130 shows a first result from the lung 10 (referring to FIG. 1) after the user interface 140 receives a first control instruction such as from a physician or nurse, and the indicator 130 shows a second result from the heart 20 (referring to FIG. 1) after the user interface 140 receives a second control instruction. Specifically, the user can input the instructions of showing the results of lungs 10 or heart 20 (referring to FIG. 1) on the user interface 140 to select the type of output on the indicator 130. Also for example, the indicator 130 shows a result of lungs if the user inputs an instruction of showing the condition of lungs 10 (referring to FIG. 1) via the user interface 140. On the other hand, the indicator 130 shows a result of a heart 20 (referring to FIG. 1) if the user inputs an instruction of showing the condition of the heart via the user interface 140. It should be noted that the memory 126 here stores the plurality of digital signals in order to compare with the received digital sound signals, but the present application is not limited thereto.

In addition, the memory 126 can store historical data according to patients' information, such as prior sensing results (digital sound signals), along with name or medical record number (MRN). Specifically, after receiving sound signals from a patient, the received sound signals can be processed by the processor 125 and stored as part of the patient's medical record. It should be noted that a storage capacity of the memory 126 may be large enough to store sound signals both for real time analysis and continuously or periodically for later retrieval, analysis and/or downloading.

Referring to FIG. 9, the signal processor 120 may further comprises a network (or communication) interface 127, such as for communication with a larger network using any applicable communication protocol, such as wireless, Ethernet, Bluetooth, etc., as described below. The network (or communication) interface 127 is coupled to the processor 125 and may have the capability to receive or transfer sound signals real time or later from the system 100 or other devices monitoring the individual simultaneously. For example, signals such as sound signals may be received from multiple sound detection apparatuses 110, 210, for processing (as described above) using a single signal processor 120, with data compared and stored separately for each patient.

The representative embodiments allow users to detect different areas of a subject, at the same time and in real time, using a plurality of sound sensors arranged in an array, so that the representative embodiments disclose a more efficient way to detect heart or lung sounds of a human subject. In addition, the real-time detection is also provided by comparing the received sound signals with the reference data stored in the signal processor 120. Therefore, the representative embodiments do not require complex follow-up analysis by medical personnel after collecting sound signals data from human subjects, thereby providing simplified processes for detecting heart or lung sounds. Moreover, by storing the historical data in the signal processor, the representative embodiments also allow the user to view historical information and then do further comparison with the current information.

Numerous advantages of the representative embodiments are readily apparent. The representative apparatus, system and method provide for noninvasive, remote, accurate, real time monitoring of heart and lung sounds. The monitoring may be continuous or periodic. The representative apparatus and system are comparatively unobtrusive, portable, convenient and easy to use for a treating physician, a nurse, a technician, other medical personnel, while nonetheless being comparatively or sufficiently accurate to obtain meaningful results and actionable information, with a comparatively fast detection time.

As used herein, a “processor” (or “controller”) 125 may be any type of processor or controller, and may be embodied as one or more processor(s) 125 configured, designed, programmed or otherwise adapted to perform the functionality discussed herein. As the term processor or controller is used herein, a processor 125 may include use of a single integrated circuit (“IC”), or may include use of a plurality of integrated circuits or other components connected, arranged or grouped together, such as controllers, microprocessors, digital signal processors (“DSPs”), array processors, graphics or image processors, parallel processors, multiple core processors, custom ICs, application specific integrated circuits (“ASICs”), field programmable gate arrays (“FPGAs”), adaptive computing ICs, associated memory (such as RAM, DRAM and ROM), and other ICs and components, whether analog or digital. As a consequence, as used herein, the term processor or controller should be understood to equivalently mean and include a single IC, or arrangement of custom ICs, ASICs, processors, microprocessors, controllers, FPGAs, adaptive computing ICs, or some other grouping of integrated circuits which perform the functions discussed herein, with associated memory, such as microprocessor memory or additional RAM, DRAM, SDRAM, SRAM, MRAM, ROM, FLASH, EPROM or EPROM. A processor 125, with associated memory, may be adapted or configured (via programming, FPGA interconnection, or hard-wiring) to perform the methodology of the invention, as discussed herein. For example, the methodology may be programmed and stored, in a processor 125 with its associated memory (and/or memory 126) and other equivalent components, as a set of program instructions or other code (or equivalent configuration or other program) for subsequent execution when the processor 125 is operative (i.e., powered on and functioning). Equivalently, when the processor 125 may implemented in whole or part as FPGAs, custom ICs and/or ASICs, the FPGAs, custom ICs or ASICs also may be designed, configured and/or hard-wired to implement the methodology of the invention. For example, the processor 125 may be implemented as an arrangement of analog and/or digital circuits, controllers, microprocessors, DSPs and/or ASICs, collectively referred to as a “processor” or “controller”, which are respectively hard-wired, programmed, designed, adapted or configured to implement the methodology of the invention, including possibly in conjunction with a memory 126.

The memory 126, which may include a data repository (or database), may be embodied in any number of forms, including within any computer or other machine-readable data storage medium, memory device or other storage or communication device for storage or communication of information, currently known or which becomes available in the future, including, but not limited to, a memory integrated circuit (“IC”), or memory portion of an integrated circuit (such as the resident memory within a processor 125 or processor IC), whether volatile or non-volatile, whether removable or non-removable, including without limitation RAM, FLASH, DRAM, SDRAM, SRAM, MRAM, FeRAM, ROM, EPROM or EPROM, or any other form of memory device, such as a magnetic hard drive, an optical drive, a magnetic disk or tape drive, a hard disk drive, other machine-readable storage or memory media such as a floppy disk, a CDROM, a CD-RW, digital versatile disk (DVD) or other optical memory, or any other type of memory, storage medium, or data storage apparatus or circuit, which is known or which becomes known, depending upon the selected embodiment. The memory 126 may be adapted to store various look up tables, parameters, coefficients, other information and data, programs or instructions (of the software of the present invention), and other types of tables such as database tables.

As indicated above, the processor 125 is hard-wired or programmed, using software and data structures of the invention, for example, to perform the methodology of the present invention. As a consequence, the system and related methods of the present invention may be embodied as software which provides such programming or other instructions, such as a set of instructions and/or metadata embodied within a non-transitory computer readable medium, discussed above. In addition, metadata may also be utilized to define the various data structures of a look up table or a database. Such software may be in the form of source or object code, by way of example and without limitation. Source code further may be compiled into some form of instructions or object code (including assembly language instructions or configuration information). The software, source code or metadata of the present invention may be embodied as any type of code, such as C, C++, Matlab, SystemC, LISA, XML, Java, Brew, SQL and its variations (e.g., SQL 99 or proprietary versions of SQL), DB2, Oracle, or any other type of programming language which performs the functionality discussed herein, including various hardware definition or hardware modeling languages (e.g., Verilog, VHDL, RTL) and resulting database files (e.g., GDSII). As a consequence, a “construct”, “program construct”, “software construct” or “software”, as used equivalently herein, means and refers to any programming language, of any kind, with any syntax or signatures, which provides or can be interpreted to provide the associated functionality or methodology specified (when instantiated or loaded into a processor or computer and executed, including the processor 125, for example).

The software, metadata, or other source code of the present invention and any resulting bit file (object code, database, or look up table) may be embodied within any tangible, non-transitory storage medium, such as any of the computer or other machine-readable data storage media, as computer-readable instructions, data structures, program modules or other data, such as discussed above with respect to the memory 126, e.g., a floppy disk, a CDROM, a CD-RW, a DVD, a magnetic hard drive, an optical drive, or any other type of data storage apparatus or medium, as mentioned above.

The network (or communication) interface 127 are utilized for appropriate connection to a relevant channel, network or bus; for example, the network (or communication) interface 127 may provide impedance matching, drivers and other functions for a wireline or wireless interface, may provide demodulation and analog to digital conversion for a wireless interface, and may provide a physical interface, respectively, for the processor 125 and/or memory 126, with other devices. In general, the network (or communication) interface 127 are used to receive and transmit data, depending upon the selected embodiment, such as program instructions, parameters, configuration information, control messages, data and other pertinent information.

The various optional filter(s) 122, amplifier(s) 123, and one or more A/D converters 124 all may be implemented as known or may become known in the art.

The network (or communication) interface 127 may be implemented as known or may become known in the art, to provide data communication between the processor 125 and any type of network or external device, such as wireless, optical, or wireline, and using any applicable standard (e.g., one of the various PCI, USB, RJ 45, Ethernet (Fast Ethernet, Gigabit Ethernet, 300ase-TX, 300ase-FX, etc.), IEEE 802.11, Bluetooth, WCDMA, WiFi, GSM, GPRS, EDGE, 3G and the other standards and systems mentioned above, for example and without limitation), and may include impedance matching capability, voltage translation for a low voltage processor to interface with a higher voltage control bus, wireline or wireless transceivers, and various switching mechanisms (e.g., transistors) to turn various lines or connectors on or off in response to signaling from processor 125. In addition, the network (or communication) interface 127 may also be configured and/or adapted to receive and/or transmit signals externally to the system 100, such as through hard-wiring or RF or infrared signaling, for example, to receive information in real-time for output on a display, for example. The network (or communication) interface 127 may provide connection to any type of bus or network structure or medium, using any selected architecture. By way of example and without limitation, such architectures include Industry Standard Architecture (ISA) bus, Enhanced ISA (EISA) bus, Micro Channel Architecture (MCA) bus, Peripheral Component Interconnect (PCI) bus, SAN bus, or any other communication or signaling medium, such as Ethernet, ISDN, T1, satellite, wireless, and so on.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention. One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. In addition, every intervening sub-range within range is contemplated, in any combination, and is within the scope of the disclosure. For example, for the range of 5-10, the sub-ranges 5-6, 5-7, 5-8, 5-9, 6-7, 6-8, 6-9, 6-10, 7-8, 7-9, 7-10, 8-9, 8-10, and 9-10 are contemplated and within the scope of the disclosed range.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical, structural or magnetic coupling, connection or attachment, or adaptation or capability for such a direct or indirect electrical, structural or magnetic coupling, connection or attachment, including integrally formed components and components which are coupled via or through another component.

With respect to signals, we refer herein to parameters that “represent” a given metric or are “representative” of a given metric, where a metric is a measure of a state of at least part of the regulator or its inputs or outputs. A parameter is considered to represent a metric if it is related to the metric directly enough that regulating the parameter will satisfactorily regulate the metric. A parameter may be considered to be an acceptable representation of a metric if it represents a multiple or fraction of the metric.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

What is claimed is:

1. A sound detection apparatus for detecting heart or lung sounds of a human subject, comprising:
a flexible material layer; and
a plurality of sound sensors coupled to the flexible material layer.

2. The sound detection apparatus of claim 1, further comprising:
a signal output interface coupled to the plurality of sound sensors.

3. The sound detection apparatus of claim 2, wherein the signal output interface comprises:
a signal output coupling; and
a plurality of wires coupled to the plurality of sound sensors and to the signal output coupling.

4. The sound detection apparatus of claim 1, further comprising:
an adhesive layer coupled to the flexible material layer.

5. The sound detection apparatus of claim 1, wherein the plurality of sound sensors are arranged in an array on or within the flexible material layer.

6. The sound detection apparatus of claim 5, wherein the array of the plurality of sound sensors has an X-shaped configuration of the plurality of sound sensors or symmetrical inverted Y-shaped configurations of the plurality of sound sensors.

7. The sound detection apparatus of claim 1, wherein the flexible material layer has a flat form factor and is dimensioned to correspond to a size of the human subject.

8. The sound detection apparatus of claim 1, wherein the flexible material layer is X-shaped.

9. The sound detection apparatus as of claim 1, wherein the flexible material layer is wearable.

10. A sound detection apparatus for detecting heart or lung sounds of a human subject, comprising:
a plurality of sound sensors arranged in an array; and
a signal output interface coupled to the plurality of sound sensors.

11. The sound detection apparatus of claim 10, wherein the signal output interface comprises:
a signal output coupling; and
a plurality of wires coupled to the plurality of sound sensors and to the signal output coupling.

12. The sound detection apparatus of claim 11, further comprising:
a housing comprising a flexible material layer, wherein the plurality of sound sensors are arranged in the array on or within the flexible material layer, and wherein the plurality of wires are arranged on or within the flexible material layer.

13. The sound detection apparatus of claim 12, wherein the flexible material layer has a flat form factor and is dimensioned to correspond to a size of the human subject.

14. The sound detection apparatus of claim 10, wherein the array of the plurality of sound sensors has an X-shaped configuration of the plurality of sound sensors or symmetrical inverted Y-shaped configurations of the plurality of sound sensors.

15. A sound detection system for detecting heart or lung sounds of a human subject, comprising:
a sound detection apparatus comprising:
a flexible material layer;
an array of a plurality of sound sensors coupled to the flexible material layer, the plurality of sound sensors to generate a plurality of sound signals, and
a signal output interface coupled to the plurality of sound sensors;
an indicator; and
a signal processor coupled to the signal output interface and to the indicator, the signal processor comprising:
a memory storing reference data; and
a processor coupled to the indicator, to the memory and to the plurality of sound sensors, the processor adapted to receive the plurality of sound signals, to compare the received plurality of sound signals with the reference data, and when a variance or difference between one or more of the received plurality of sound signals and the reference data is greater than a predetermined threshold, to generate a corresponding command signal to the indicator to provide a warning signal indication.

16. The sound detection system of claim 15, wherein the signal processor further comprises:
a filter coupled to the plurality of sound sensors to generate a plurality of filtered sound signals;
an amplifier coupled to the filter to receive the plurality of filtered sound signals and generate a plurality of amplified sound signals; and
an analog-to-digital converter coupled to the processor and coupled to the amplifier, the analog-to-digital converter to receive the plurality of amplified sound signals and generate a plurality of digital sound signals and provide the plurality of digital sound signals to the processor to form the received plurality of sound signals.

17. The sound detection system of claim 16, wherein the memory further stores the plurality of digital sound signals.

18. The sound detection system of claim 15, wherein the indicator is a visual display.

19. The sound detection system of claim 15, wherein the indicator comprises a visual monitor to display an image of a detected area.

20. The sound detection system of claim 15, wherein the indicator comprises an auditory speaker to generate a warning sound.

21. The sound detection system of claim 15, further comprising:
a user interface coupled to the processor, the user interface to receive user input;
wherein the indicator is adapted to display one or more different types of output in response to user input.

22. A method for detecting heart or lung sounds of a human subject, comprising:
using a sensor array, detecting a plurality of sounds from a predetermined region of the human subject;
generating a plurality of sound signals in response to the detected plurality of sounds;
using a processor, comparing the plurality of sound signals to reference data; and
generating a warning signal indication when a variance or difference between the plurality of sound signals and the reference data is greater than a predetermined threshold.

23. The method of claim 22, further comprising:
filtering the plurality of sound signals;
amplifying the filtered plurality of sound signals; and
converting the amplified plurality of sound signals to a plurality of digital sound signals.
